(19) United States
(12) Patent Application Publication
(10) Pub. No.: US 2017/0327375 A1
(43) Pub. Date: Nov. 16, 2017

(54) Short-Chain Potassium Polyphosphate Compositions and Methods of Making

(71) Applicant: ICL Performance Products LP, St. Louis, MO (US)

(72) Inventor: David Richard Gard, St. Louis, MO (US)

(21) Appl. No.: 15/591,557

(22) Filed: May 10, 2017

Related U.S. Application Data
(60) Provisional application No. 62/334,541, filed on May 11, 2016.

(51) Int. Cl. C01B 25/41; A61K 8/24; C08G 79/04; C01B 25/12

(52) U.S. Cl. CPC C01B 25/41; A61K 8/24; C08G 79/04; C01B 25/12

(57) ABSTRACT

Disclosed herein are methods of making acidic short-chain potassium polyphosphate compositions with reduced levels of water-insoluble materials. Also disclosed herein are acidic short-chain potassium polyphosphate compositions.

### CROSS REFERENCE TO RELATED APPLICATIONS

This application is a non-provisional U.S. patent application that claims the benefit of U.S. Provisional Patent Application No. 62/334,541, filed May 11, 2016, the entirety of which is hereby incorporated by reference.

### BACKGROUND

Sodium reduction is an industry-wide goal for the formulation of food and related products. Despite their potential for this, and use in many other applications, short-chain acidic potassium polyphosphates (e.g., potassium acid pyrophosphate (KAPP)) have generally not been commercially available because of difficulties in preparing them without significant levels of impurities.

Acidic polyphosphates can be prepared by the partial thermal dehydration of orthophosphates. Compositions prepared by the thermal route (i.e., heating potassium orthophosphate) contain significant quantities of water-insoluble potassium metaphosphate and/or unreacted orthophosphate. Once conversion of the orthophosphate reaches ˜70% (and sometimes lower), insoluble potassium metaphosphate rises significantly to 1-2% or more. Thus, to maintain no more than ˜1% insoluble potassium metaphosphate in the product, ≧30% of the P₂O₅ content of the product remains in the form of orthophosphate. Orthophosphate exhibits different functionalities than pyrophosphate and other short-chain polyphosphates—for example, especially with respect to chelating ability. Insoluble potassium metaphosphate must either be removed or tolerated in the otherwise highly water-soluble product.

KAPP can be prepared by the partial thermal dehydration of monopotassium phosphate (MKP), for example:

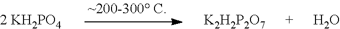

This reaction is accompanied by the formation of insoluble potassium metaphosphate:

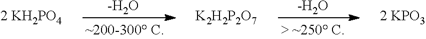

As a result, preparation of KAPP by thermal dehydration of MKP results in a product containing significant amounts of MKP (under-converted raw material) and/or insoluble metaphosphate (over-converted material).

The potassium system behaves differently from the analogous sodium system, wherein sodium acid pyrophosphate (SAPP) can be produced in high assay by the thermal dehydration of monosodium phosphate.

Thus, there remains a need to develop short-chain acidic potassium polyphosphate compositions and methods of making them in a manner more suitable for commercial exploitation.

### SUMMARY

An aspect of the present invention includes a potassium polyphosphate composition. This potassium polyphosphate composition includes a mixture of acidic potassium polyphosphates, wherein said mixture of acidic potassium polyphosphates comprises from about 1% to about 30% of its P₂O₅ content as orthophosphate and from about 1% to about 20% of its P₂O₅ content as tetrapoly- and higher polyphosphates, and wherein said composition is a solid composition of matter.

Another aspect of the present invention includes a method of preparing a potassium polyphosphate composition, the method comprising reacting a polyphosphoric acid in the range of from about 75 wt % to about 86 wt % P₂O₅ and a potassium polyphosphate salt to form a reaction mixture.

These are merely some of the innumerable aspects of the present invention and should not be deemed an all-inclusive listing of the innumerable aspects associated with the present invention.

### BRIEF DESCRIPTION OF THE DRAWINGS

FIG. 1 is a graphical representation of results of a “comparative example” of the thermal method of converting MKP to KAPP showing that the formation of water-insoluble materials disadvantageously increases rapidly as the rate of conversion increases.

### DETAILED DESCRIPTION

To the extent necessary to provide descriptive support, the subject matter and/or text of the appended claims is incorporated herein by reference in their entirety. It will be understood by all readers of this written description that the exemplary embodiments described and claimed herein may be suitably practiced in the absence of any recited feature, element or step that is, or is not, specifically disclosed herein.

### Definitions

It is to be noted that the term “a” or “an” entity refers to one or more of that entity; for example, “a polyphosphate,” is understood to represent one or more polyphosphates. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

All methods described herein can be performed in any suitable order unless otherwise indicated herein. No language or terminology in this specification should be construed as indicating any non-claimed element as essential or critical.

Unless defined otherwise, technical and scientific terms used herein have the same meaning as commonly understood by one of ordinary skill in the art to which this disclosure is related.

Concentrations, amounts, and other numerical data may be presented here in a range format (e.g., from 5% and 20%). It is to be understood that such range format is used merely for convenience and brevity and should be interpreted flexibly to include not only the numerical values explicitly recited as the limits of the range, but also to include all the individual numerical values or sub-ranges encompassed within that range, as if each numerical value and sub-range is explicitly recited. For example, a range of from 5% to 20% should be interpreted to include numerical values such as, but not limited to, 5%, 5.5%, 9.7%, 10.3%, 15%, etc., and sub-ranges such as, but not limited to, 5% to 10%, 10% to 15%, 8.9% to 18.9%, etc. Numeric ranges are inclusive of the numbers defining the range.

Units, prefixes, and symbols are denoted in their Systeme International de Unites (SI) accepted form.

The headings provided herein are not limitations of the various aspects or aspects of the disclosure, which can be had by reference to the specification as a whole.

As used herein, a “solid composition of matter” is a material that exhibits a viscosity greater than or equal to 0.1 kPa·sec. Generally, a composition that is considered completely solid, exhibits a viscosity greater than 100 kPa·sec. As used herein, a “semi-solid” exhibits a viscosity in the range of 0.1 kPa·sec to 100 kPa·sec. As used herein, a “paste” exhibits a viscosity in the range of 0.1 kPa·sec to 1.0 kPa·sec.

As used herein, “water insoluble content,” “insolubles,” “water-insolubles,” “water-insoluble compounds,” and the like refer to the residual undissolved solids remaining after dissolving 10 grams of the potassium polyphosphate composition in 100 milliliters of water at 20 to 25° C. “Short-chain” is defined as pyrophosphate and tripolyphosphate.

### Overview

Acidic short-chain potassium polyphosphates, such as potassium acid pyrophosphate (KAPP), have been absent from commercial phosphate portfolios because of the difficulties in making them without significant quantities of orthophosphates and/or insoluble potassium metaphosphate as impurities. Disclosed herein are methods of making acidic short-chain potassium polyphosphate compositions with reduced levels of these impurities. Also disclosed herein are acidic short-chain potassium polyphosphate compositions. In certain embodiments, the compositions are solid compositions of matter.

Certain compositions disclosed herein can be water-soluble solid, acidic, short-chain polyphosphate mixtures that are essentially free of water-insoluble material. Applications can include use as an acidifier, pH control agent, buffering agent, and chelating agent. As a potassium analogue of sodium acid pyrophosphate (SAPP), KAPP in particular may have utility as a no-sodium alternative to SAPP. Certain compositions disclosed herein thus can find use in foods (e.g., leavening, meats, poultry, produce, potatoes, dairy, cheese, and beverages), in companion animal (pet) foods, and in dental preparations. Uses can also include, for example, water treatment and as a corrosion inhibitor, stain remover, dispersant for oil well drilling muds, and as an acid cleaner.

Processes described herein comprise reacting a polyphosphoric acid and a potassium polyphosphate salt to form a reaction mixture. Further, it has been unexpectedly discovered that water can serve to increase the level of pyrophosphate and reduce the level of orthophosphate during the preparation of short-chain potassium polyphosphates. Thus, in certain embodiments, water is added to the system. It has also been discovered that the resultant phosphate species distributions are significantly different from the expected distributions (i.e., the “combined composition” based on the combination of polyphosphoric acid and potassium polyphosphate salt prior to reaction), and that such resultant distributions are dependent on the conditions for acid dilution, mixing (reaction), and/or drying. It has also been discovered that tripolyphosphate and higher species can be collectively reduced in the course of processing while the levels of both pyrophosphate and orthophosphate are typically increased. In certain embodiments, provided that the starting acid concentration and the added water content are high enough, the processing time is short enough, the pH is high enough, the pressure over the drying operation is low enough, and/or that the drying temperature is low enough, the content of the short-chain polyphosphates (total pyro- and tripolyphosphate) can be increased above that from the “combined composition” while maintaining an orthophosphate content below about 30%.

Bound by theory, in the absence of added water, the reaction mixture is believed to be essentially an anhydrous system although water may not be completely absent. The raw materials are essentially anhydrous; free water is thought to be absent in polyphosphoric acid at concentrations greater than about 80.5% P₂O₅ (A. L. Huhti and P. A. Gartaganis, The Composition of the Strong Phosphoric Acids, Can. J. Chem. 34, 785 (1956)). Water is neither generated nor consumed in the reaction between polyphosphoric acid and potassium polyphosphate salt. The lower average chain length in some embodiments for the reaction products produced without added water, when compared to the average chain length for the corresponding “combined composition,” was unexpected.

The addition of water to the polyphosphoric acid, the potassium polyphosphate salt, and/or the reaction mixture results in overall hydrolytic degradation, as observed by a decrease in the average chain length compared to that of the “combined composition.” As the level of added water is increased, it would be expected that the average chain length of the reaction product would decrease. Although a minimum in average chain length was generally observed at around 4.4% added water, in certain embodiments, it was unexpectedly observed that the average chain length increased with increasing levels of added water.

### Compositions

Certain potassium polyphosphate compositions described herein are essentially free of water-insoluble impurities and contain lower orthophosphate levels than generally possible using the thermal method of production. Certain embodiments contain primarily short chain acidic potassium polyphosphates, such as KAPP.

In certain embodiments, a potassium polyphosphate composition comprises a mixture of acidic potassium polyphosphates (it will be understood that as used herein a “mixture of acidic potassium polyphosphates” can comprise orthophosphate in addition to multiple potassium polyphosphates). Certain embodiments comprise hydrates and double salts of the polyphosphates. For example but not limited to K₂H₂P₂O₇.0.5H₂O (hydrate) and 2KH₂PO₄.K₂H₂P₂O₇ (double salt). In certain embodiments, the mixture of acidic potassium polyphosphates is a solid composition of matter. In certain embodiments, it comprises from about 1% to about 30% of its P₂O₅ content as orthophosphate and from about 1% to about 20% of its P₂O₅ content as tetrapoly- and higher polyphosphates. Thus, in certain embodiments, the mixture comprises from any of about 1%, 2%, 3%, 4%, 5%, 10%, 15%, 20%, 25%, 26%, 27%, 28%, or 29% to any of about 2%, 3%, 4%, 5%, 10%, 15%, 20%, 25%, 26%, 27%, 28%, 29%, or 30% of its P₂O₅ content as orthophosphate and from any of about 1%, 2%, 3%, 4%, 5%, 10%, 15%, 16%, 17%, 18%, or 19% to any of about 2%, 3%, 4%, 5%, 10%, 15%, 16%, 17%, 18%, 19% or 20% (where applicable) of its P₂O₅ content as tetrapoly- and higher polyphosphates. In certain embodiments, the mixture of acidic potassium polyphosphates comprises from about 50% to about 95% of its P₂O₅ content as pyrophosphate. In certain embodiments, the mixture of acidic potassium polyphosphates comprises from about 50% to about 82% of its P₂O₅ content as pyrophosphate. Thus, in certain embodiments, the mixture of acidic potassium polyphosphates comprises from any of about 50%, 51%, 52%, 53%, 54%, 55%, 56%, 57%, 58%, 59%, 60%, 65%, 70%, 75%, 80%, 81%, 82%, 83%, 84%, 85%, 86%, 87%, 88%, 89%, 90%, 91%, 92%, 93%, or 94% to any of about 51%, 52%, 53%, 54%, 55%, 56%, 57%, 58%, 59%, 60%, 65%, 70%, 75%, 80%, 81%, 82%, 83%, 84%, 85%, 86%, 87%, 88%, 89%, 90%, 91%, 92%, 93%, 94%, or 95% of its P₂O₅ content as pyrophosphate. In certain embodiments, the mixture of acidic potassium polyphosphates comprises greater than about 90% of its P₂O₅ content as pyrophosphate.

In certain embodiments, the mixture of acidic potassium polyphosphates comprises from about 2% to about 15% of its P₂O₅ content as tripolyphosphate. Thus, in certain embodiments, the mixture of acidic potassium polyphosphates comprises from any of about 2%, 3%, 4%, 5%, 6%, 7%, 8%, 9%, 10%, 11%, 12%, 13%, or 14% to any of about 3%, 4%, 5%, 6%, 7%, 8%, 9%, 10%, 11%, 12%, 13%, 14%, or 15% of its P₂O₅ content as tripolyphosphate.

In certain embodiments, the combined amount of pyrophosphate and tripolyphosphate in the mixture of acidic potassium polyphosphates is from about 50% to about 97% of the P₂O₅ content of the mixture. Thus, in certain embodiments, the mixture of acidic potassium polyphosphates comprises from any of about 50%, 55%, 60%, 65%, 70%, 75%, 80%, 85%, 90%, or 95% to any of about 55%, 60%, 65%, 70%, 75%, 80%, 85%, 90%, 95%, or 97% of its P₂O₅ content as pyrophosphate and/or tripolyphosphate.

In certain embodiments, a potassium polyphosphate composition comprising a mixture of acidic potassium polyphosphates is essentially free of water-insoluble impurities. In certain embodiments, the potassium polyphosphate composition has a low water-insoluble content that does not exceed about 0.01%, 0.1%, 0.2%, 0.3%, 0.4%, 0.5%, 0.6%, 0.7%, 0.8%, 0.9%, 1.0%, 1.1%, 1.2%, 1.3%, 1.4%, or 1.5%. Thus, in certain embodiments, the water-insoluble content of the composition is from any of about 0.0%, 0.001%, 0.01%, 0.1%, 0.2%, 0.3%, 0.4%, 0.5%, 0.6%, 0.7%, 0.8%, 0.9%, 1.0%, 1.1%, 1.2%, 1.3%, or 1.4% to any of about 0.001%, 0.01%, 0.1%, 0.2%, 0.3%, 0.4%, 0.5%, 0.6%, 0.7%, 0.8%, 0.9%, 1.0%, 1.1%, 1.2%, 1.3%, 1.4%, or 1.5%. In certain embodiments, the low water-insoluble content described herein is achieved without the need to filter the composition to remove water-insoluble material or otherwise treat the composition to remove water-insoluble material.

In certain embodiments, a potassium polyphosphate composition comprising a mixture of acidic potassium polyphosphates comprises a K₂O/P₂O₅ molar ratio of from about 0.8 to about 1.2, or from about 0.9 to about 1.1, or from about 0.95 to about 1.05. Thus, in certain embodiments, the K₂O/P₂O₅ mole ratio is from any of about 0.8, 0.9, 0.95, 1.0, 1.05, or 1.1, to any of about 0.9, 0.95, 1.0, 1.05, 1.1, or 1.2. An overall K₂O/P₂O₅ mole ratio near 1.0 yields a mixture of polyphosphates having a general formula approximating KₙH₂PₙO₍₃ₙ₊₁₎.

In certain embodiments, a potassium polyphosphate composition comprising a mixture of acidic potassium polyphosphates comprises hydrates and double salts of the polyphosphates. The presence of or existence as double salts or hydrates can be determined by powder x-ray diffraction. Hydrates can also be determined by thermogravimetric analysis (TGA).

In certain embodiments, a potassium polyphosphate composition comprising a mixture of acidic potassium polyphosphates has an average chain length (n) of between about 1.5 to about 3.5, between about 1.7 to about 3.3, between about 1.7 to about 3.0, or between about 1.7 to about 2.8. Thus, in certain embodiments, the average chain length (n) of from any of about 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, or 3.4 to any of about 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, or 3.5.

In certain embodiments of a potassium polyphosphate composition comprising a mixture of acidic potassium polyphosphates, a 1% by weight solution of the composition in water has a pH of from any of about pH 3, pH 4, pH 5, or pH 6 to any of about pH 4, pH 5, pH 6 or pH 7. In certain embodiments, a 1% by weight solution of the composition in water has a pH of from about pH 4 to about pH 5.

### Methods of Preparation

Certain disclosed methods provide for the production of potassium polyphosphates with a high content of short-chain polyphosphates, low orthophosphate content, and essentially no water-insoluble material, including any of the compositions described herein. Such compositions are believed to be unobtainable by thermal methods and otherwise difficult to obtain. It has been discovered and demonstrated that certain processing conditions result in the enhancement of pyrophosphate at the expense of longer chain polyphosphates while at the same time limiting the formation of orthophosphate and/or water-insoluble materials.

Certain embodiments are drawn to methods of preparing a potassium polyphosphate composition (such as KAPP), wherein a polyphosphoric acid and a potassium phosphate salt are mixed/reacted to form a reaction mixture. In certain embodiments, the reaction mixture is then dried and in certain embodiments, a solid composition of matter is formed. In certain embodiments, the polyphosphoric acid is in the range of from about 75 wt % to about 86 wt % P₂O₅. Representative examples of useful potassium polyphosphate salts include tetrapotassium pyrophosphate (TKPP, K₄P₂O₇), potassium tripolyphosphate (KTPP, K₅P₃O₁₀), or a mixture thereof. In certain embodiments, the potassium polyphosphate salt is tetrapotassium pyrophosphate (TKPP, K₄P₂O₇).

Degradation of polyphosphates is a function of pH, time, pressure, and/or temperature. For example, shorter times can allow for higher temperatures and low temperatures can allow for longer times. Also, for example, lower pressure allows for drying at lower temperature and/or within a shorter period of time. Thus, generally for example, drying at 80° C. under vacuum produces a certain product while drying at 80° C. under ambient pressure drying would not produce a product with the same polyphosphate species distribution. It is understood that wherein one parameter value of pH, time, pressure, or temperature is described, any other parameter value(s) disclosed herein may be used in combination. In certain embodiments, the step of drying the reaction mixture is performed at a temperature range from about 0° C. to about 100° C., at a pressure range from about 1 kPa to about 101 kPa, at a pressure ranging from a complete vacuum to about ambient atmospheric pressure, and/or for a duration of time from about 1 second to about 24 hours. In certain embodiments, the step of drying the reaction mixture is performed at a temperature range from about 40° C. to about 60° C., or about 45° C. to about 55° C., or about 60° C. to about 100° C., or about 70° C. to about 90° C., or about 75° C. to about 85° C. In certain embodiments, the step of drying the reaction mixture is performed at a temperature of about 50° C. or about 80° C. In certain embodiments, the step of drying the reaction mixture is performed under a vacuum. In certain embodiments, the step of drying the reaction mixture is performed at ambient atmospheric pressure. In certain embodiments, the step of drying the reaction mixture is performed at a combination of pressure and temperature of ≦5 kPa and ≦100° C., or ≦10 kPa and ≦80° C., or ≦101 kPa and ≦60° C. In certain embodiments, the duration of drying is that necessary to essentially remove water in a molecular form, i.e., all water not present as hydrate water or constituting acid groups of the phosphates. Water in a molecular form, also referred to as “free water”, is essentially removed when reaction mixtures are dried to a constant weight loss at temperatures and pressures such as that specified above. Throughout this patent application, experimentally all samples were dried to a constant weight loss. Therefore, the samples were dried for a long enough of a time that even longer drying times result in no additional weight loss (i.e. water loss).

In certain embodiments, the reaction mixture of the polyphosphoric acid and potassium polyphosphate salt is an essentially anhydrous system. In certain embodiments, the reaction mixture of the polyphosphoric acid and potassium polyphosphate salt is free or essentially free of water not present as hydrate water or constituting acid groups of the phosphates. In certain embodiments, the reaction mixture of the polyphosphoric acid and potassium polyphosphate salt comprises water. In certain embodiments, water is a component of the polyphosphoric acid and/or the potassium polyphosphate salt composition. In certain embodiments, water can be added to the polyphosphoric acid, potassium polyphosphate salt, or the reaction mixture of the two. In certain embodiments, the method comprises adding water. In certain embodiments, the amount of water is from about 0.1% to about 95% by weight, from about 0.1% to about 40% by weight, from about 0.1% to about 30% by weight, from about 20% to about 95% by weight, from about 20% to about 40% by weight, from about 20% to about 30% by weight, or from about 30% to about 40% by weight of the total reaction mixture after the addition of water (but before drying). Thus, in certain embodiments, the amount of water is from about any of 0.1%, 1.0%, 5%, 10%, 20%, 30%, or 40% to about any of 1.0%, 5%, 10%, 20%, 30%, 40%, 50%, 60%, 70%, 80%, 90%, or 95% by weight of the total reaction mixture after the addition of water.

In the “Direct Mix” method (see, e.g., Example B below), polyphosphoric acid and a potassium polyphosphate salt are mixed together, optionally with a small amount of water, such that the resultant reaction mixture is a semi-solid or paste. The level of water in the reaction mixture is typically less than about 30% of the total reaction mixture after the addition of water.

In certain embodiments, water can be added to the polyphosphoric acid before it is mixed/reacted with the potassium polyphosphate salt, although this is not a requirement. In certain embodiments, the water, polyphosphoric acid, and potassium polyphosphate salt can be combined in any order. In certain embodiments, the water, polyphosphoric acid, and potassium polyphosphate salt can be combined simultaneously or relatively simultaneously (i.e., all combined together within a few seconds to a few minutes to a few hours). The physical characteristics of the reaction mixture can differ depending on the order of addition of the acid, potassium salt, and water. In particular, if a relatively small amount of water is employed, a potassium salt such as TKPP may hydrate and remain as a solid phase if the acid is added afterwards. In this example, the reaction mixture will be granular (e.g., more solid, higher viscosity) before drying. In certain embodiments, once water and polyphosphoric acid come into contact they are reacted with the potassium polyphosphate salt within a given amount of time. In certain embodiments, once water is added to the polyphosphoric acid, this mixture is reacted with the potassium polyphosphate salt within about 1 hour, within about 45 minutes, within about 30 minutes, within about 20 minutes, within about 15 minutes, within about 10 minutes, within about 5 minutes, within about 4 minutes, within about 3 minutes, within about 2 minutes, or within about 1 minute. In certain embodiments, the mixture of polyphosphoric acid and water can be cooled to negate the heat of dilution. For example, with a stirred reaction or a mixer equipped with a jacket or tubing through which a coolant fluid is passed for the purpose of heat transfer. The coolant does not come into direct contact with the reaction mixture but removes heat from the reaction. On a smaller scale, a mixture can be cooled by placing a beaker, such as a stirred beaker, in a cold water or ice bath.

In certain embodiments, once water is added to the polyphosphoric acid, it is reacted with the potassium polyphosphate salt within a short enough period of time so as not to permit excessive polyphosphate hydrolysis. A significant degree of hydrolysis, however, can occur. In certain embodiments, this can be as high as about 71%. Without being bound by theory, it is believed that the rate at which phosphoric acid is hydrolyzed by added water is fast enough that the phosphoric acid species distribution of the diluted acid can be significantly affected by the temperature of the acid/water mixture, and by the time that the acid/water mixture is held before reacting with tetrapotassium pyrophosphate. As a result, the phosphate species distribution of the potassium acid pyrophosphate product can be significantly affected as well. For this reason, in certain embodiments, the temperature for the dilution of the polyphosphoric acid is controlled and/or the diluted acid must be reacted with the potassium polyphosphate salt within a short enough period of time so as not to permit excessive polyphosphate hydrolysis. Only a relatively small proportion of added water has the potential to quickly and significantly degrade the polyphosphoric acid prior to reaction with the potassium polyphosphate. For example, in the preparation of the reaction mixture with tetrapotassium pyrophosphate, water added at a 5% level is sufficient to convert phosphoric acid at 79.2% P₂O₅ completely to orthophosphoric acid. Likewise, water added at a 7% level is sufficient to convert phosphoric acid at 84.5% P₂O₅ completely to orthophosphoric acid. Thus, certain embodiments aim to minimize the reaction between acid and water before the reaction of acid with potassium polyphosphate salt can occur.

Should the acid/water mixture come to equilibrium prior to reaction with tetrapotassium pyrophosphate, the reaction products will contain up to at least half of the total P₂O₅ as orthophosphate. Thus, in certain embodiments, it is important to limit the degree of hydrolysis by maintaining low temperatures and short processing times, especially for mixtures containing polyphosphoric acid and water. Given the low pH of the system, some degree of hydrolysis is unavoidable. Some hydrolysis is acceptable for the conversion of tetrapolyphosphate and higher species to pyrophosphate. However hydrolysis that is too extensive cannot be tolerated as the phosphate species distribution will skew towards undesirably high levels of orthophosphate. The allowable limit to the extent of hydrolysis during acid dilution is difficult to determine precisely because it depends on many factors including the subsequent reaction and drying steps. However, the presence of orthophosphate for the entire product should not exceed 30% of the total P₂O₅ as orthophosphate.

In certain embodiments, some or all of the added water can come from the potassium polyphosphate salt solution that is reacted directly with the polyphosphoric acid.

In certain embodiments, sufficient water is added to the polyphosphoric acid and potassium polyphosphate salt such that the resultant mixture is, for example, a solution or a slurry (referred to herein as “Wet Mix”; see Example C below). In these embodiments, the level of water may exceed 30% by weight of the total reaction mixture, and the solution or slurry may be flowable/pumpable. The level of water is typically less than about 30% by weight of the total reaction mixture but, in certain embodiments, can exceed 30% by weight of the total reaction mixture.

Following the reaction, the mixture can be dried and/or the solids can be separated from the mother liquor by, for example, filtration, and/or centrifugation. The resultant phosphate species distributions are significantly different from the expected distribution (i.e., the “combined composition” based on the reaction between polyphosphoric acid and potassium polyphosphate salt), and is dependent on the conditions for acid dilution, mixing (reaction), drying, and if applicable, separation. It has been discovered that in certain embodiments, tripolyphosphate and higher species are collectively reduced in the course of processing. The levels of both pyrophosphates and orthophosphates are typically increased. Again, regarding hydrolysis of the polyphosphoric acid or of the acid/potassium polyphosphate salt mixture, extensive hydrolysis can be avoided for the Wet Mix approaches as well. Provided that the starting acid concentration and the added water content are high enough, the processing duration short enough, and/or the drying temperature low enough, the content of the short-chain polyphosphates (total pyro- and tripolyphosphate) is increased above that from the “combined composition,” while maintaining an orthophosphate content below about 30%. In certain embodiments, separation of solids from a reaction slurry results in a solid acidic potassium polyphosphate product having greater than about 90% of the total P₂O₅ as pyrophosphate and only a nominal level of orthophosphate (e.g., about 3%). The liquid separated from a reaction slurry may be recycled to create an efficient use of raw material and reduce or potentially eliminate waste.

The following examples of specific aspects are offered for illustrative purposes only, and are not intended to limit the scope of the present disclosure in any way.

### EXAMPLES

### A. Raw Materials

Compositions of the raw materials polyphosphoric acid and potassium pyrophosphate are shown in Table 1.

TABLE 1
Compositions of Polyphosphoric Acids and Tetrapotassium Pyrophosphate

| | Polyphosphoric | | | Tetrapotassium |
|---|---|---|---|---|
| | Acids | | | Pyrophosphate |
| Wt % P₂O₅ | 79.2 | 80.4 | 84.5 | 42.97 |
| Phosphate | | | | |
| Species, | | | | |
| % of Total P₂O₅ | | | | |
| P1 (ortho) | 21.7 | 13.0 | 4.1 | 0.33 |
| P2 (pyro) | 43.5 | 33.0 | 12.8 | 99.67 |
| P3 (tripoly) | 21.3 | 23.8 | 15.0 | |
| P4 (tetrapoly) | 8.8 | 14.6 | 15.0 | |
| P5 | 3.2 | 8.1 | 13.5 | |
| P6 | 1.1 | 3.9 | 10.9 | |
| P7 | 0.4 | 1.7 | 8.1 | |
| P8 | 0.2 | 0.8 | 5.3 | |
| P9 | | 0.5 | 3.8 | |
| P10 | | 0.5 | 2.9 | |
| P11 | | | 2.3 | |
| P12 | | | 2.0 | |
| P13 | | | 1.9 | |
| P14 | | | 1.4 | |
| P15 | | | 0.8 | |
| Average Chain | 2.34 | 3.01 | 5.37 | 2.00 |
| Length | | | | |
| % ≧P4 | 13.70 | 30.1 | 68.0 | 0.00 |

### B. Direct Mix

In the Direct Mix method, polyphosphoric acid and a potassium polyphosphate salt are mixed together, optionally with a small amount of water, such that the resultant reaction mixture is a semi-solid or paste. The level of water is typically less than about 30% by weight of the total reaction mixture after the water has been added but before drying.

Tables 2 through 6 are representative examples showing polyphosphoric acid at different concentrations combined with tetrapotassium pyrophosphate on an equal P₂O₅ basis (overall 1.00 K/P mole ratio) along with varying amounts of water. The resultant mixtures were dried at different temperatures, either under vacuum or at ambient pressure. The resulting phosphate species distributions can be compared against the distributions of the raw materials (Combined Composition).

TABLE 2
Acid at 79.2 wt % P₂O₅, Vacuum Drying at 80° C. (Series 028)

| | | Analyzed Compositions* | | |
|---|---|---|---|---|
| Phosphate Species, | Combined | % Added Water in Mix | | |
| % of Total P₂O₅ | Composition | 0.00% | 4.35% | 11.51% |
| P1 (ortho) | 10.95 | 37.2 | 35.6 | 15.8 |
| P2 (pyro) | 71.64 | 51.2 | 61.8 | 81.8 |
| P3 (tripoly) | 10.59 | 8.5 | 2.6 | 2.4 |
| P4 (tetrapoly) | 4.38 | 2.3 | | |
| P5 | 1.59 | 0.8 | | |
| P6 | 0.55 | | | |
| P7 | 0.20 | | | |
| P8 | 0.10 | | | |
| P9 | | | | |
| P10 | | | | |
| Average Chain | 2.17 | 1.78 | 1.67 | 1.87 |
| Length | | | | |
| % ≧P4 | 6.82 | 3.1 | | |

*As used in Tables 2 to 8, “Analyzed Compositions” refers to the phosphate species distribution as a % of the total P₂O₅ as determined by analysis of the dried product.

TABLE 3
Acid at 79.2 wt % P₂O₅, Vacuum Drying at 50° C. (Series 025, 041)

| Phosphate | | | | | | | | |
|---|---|---|---|---|---|---|---|---|
| Species, | Combined | Analyzed Compositions % Added Water in Mix | | | | | | |
| % of Total P₂O₅ | Composition | 0.00% | 4.35% | 6.11% | 11.51% | 11.51% | 16.32% | 20.64% |
| P1 (ortho) | 10.95 | 24.3 | 24.3 | 14.0 | 12.1 | 16.1 | 10.8 | 11.3 |
| P2 (pyro) | 71.64 | 60.3 | 72.9 | 77.3 | 77.6 | 81.2 | 81.0 | 80.6 |
| P3 (tripoly) | 10.59 | 11.0 | 2.7 | 6.2 | 7.4 | 2.6 | 5.7 | 6.1 |
| P4 (tetrapoly) | 4.38 | 3.6 | | 1.9 | 2.0 | | 1.7 | 1.5 |
| P5 | 1.59 | 0.8 | | 0.6 | 0.8 | | 0.9 | 0.6 |
| P6 | 0.55 | | | | | | | |
| P7 | 0.20 | | | | | | | |
| P8 | 0.10 | | | | | | | |
| P9 | | | | | | | | |
| P10 | | | | | | | | |
| Average Chain | 2.17 | 1.96 | 1.78 | 1.98 | 2.02 | 1.86 | 2.01 | 2.00 |
| Length | | | | | | | | |
| % ≧P4 | 6.82 | 4.4 | — | 2.5 | 2.8 | — | 2.6 | 2.1 |

Preparations identical to those in Tables 2 and 3 with 11.51% added water were dried at various temperatures at ambient pressure (i.e., without vacuum) (COMPARATIVE EXAMPLE, Table 4). These examples shown below all intended to distinguish the current application from the prior art. Drying our reaction mixtures in the manner of in the same manner as the prior art, i.e., 160° C. and ambient pressure, provides very different results. These examples also demonstrate that drying our reaction mixtures at higher temperatures results in hydrolytic degradation to orthophosphate. As such, they are comparative examples for drying at higher temperatures versus drying at a lower temperature under vacuum.

TABLE 4
Acid at 79.2 wt % P₂O₅ and 11.51% Added Water;
Drying at Ambient Pressure (Series 043, 075)

| | Analyzed Compositions |
|---|---|
| Drying Temperature (° C.) | Orthophosphate Content |
| 80 | 55.6 |
| 150 | 59.3 |
| 160 (6 hours) | 66.9 |
| 160 (23 hours) | 55.6 |
| 240 | 80.9 |

A Dry Mix sample similar to that described in Series 025 is prepared except that polyphosphoric acid at 84.5% P₂O₅ is used instead of 79.2%, and the reaction mixture is heated at 160° C. at ambient pressure to dry. TKPP, polyphosphoric acid at 84.5% P₂O₅, and water are mixed to form a composition having a 1.00 K/P mole ratio and 11.71% added water. The sample was dried at 160° C. at ambient pressure for 5.5 hr. 68.9% of the P₂O₅ content analyzed as orthophosphate.

Results from preceding two Paragraphs above demonstrate the difficulty in recovering a product with less than about 30% of the total P₂O₅ in the form of orthophosphate by conventional drying.

TABLE 5
Acid at 80.4 wt % P₂O₅, Vacuum Drying at 50° C. (Series 053)

| | | Analyzed Compositions % | | | |
|---|---|---|---|---|---|
| Phosphate Species, | Combined | Added Water in Mix | | | |
| % of Total P₂O₅ | Composition | 0.00% | 4.35% | 11.53% | 20.68% |
| P1 (ortho) | 6.67 | 13.2 | | 10.3 | 8.3 |
| P2 (pyro) | 66.38 | 57.7 | 66.8 | 76.5 | 75.2 |
| P3 (tripoly) | 11.90 | 14.6 | 8.8 | 7.6 | 9.7 |
| P4 (tetrapoly) | 7.30 | 8.3 | 3.0 | 2.8 | 3.8 |
| P5 | 4.05 | 4.3 | 1.0 | 1.9 | 2.1 |
| P6 | 1.95 | 1.9 | | 0.9 | 0.9 |
| P7 | 0.85 | | | | |
| P8 | 0.40 | | | | |
| P9 | 0.25 | | | | |
| P10 | 0.25 | | | | |
| Average Chain | 2.50 | 2.39 | 1.97 | 2.12 | 2.19 |
| Length | | | | | |
| % ≧P4 | 15.05 | 14.5 | 4.0 | 5.6 | 6.8 |

TABLE 6
Acid at 84.5 wt % P₂O₅, Vacuum Drying at 50° C. (Series 054)

| | | Analyzed Compositions | | | |
|---|---|---|---|---|---|
| Phosphate Species, | Combined | % Added Water in Mix | | | |
| % of Total P₂O₅ | Composition | 0.00% | 4.44% | 11.71% | 20.96% |
| P1 (ortho) | 2.22 | 6.8 | 16.8 | 11.8 | 7.0 |
| P2 (pyro) | 56.31 | 54.8 | 63.2 | 71.3 | 73.5 |
| P3 (tripoly) | 7.50 | 12.3 | 12.3 | 7.7 | 6.9 |
| P4 (tetrapoly) | 7.50 | 8.9 | 5.4 | 3.2 | 2.7 |
| P5 | 6.75 | 6.3 | 2.2 | 2.1 | 2.4 |
| P6 | 5.45 | 3.9 | | 1.3 | 2.4 |
| P7 | 4.05 | 2.1 | | 1.2 | 2.6 |
| P8 | 2.65 | 1.2 | | 1.4 | 2.4 |
| P9 | 1.90 | | | | |
| P10 | 1.45 | | | | |
| P11 | 1.15 | | | | |
| P12 | 1.00 | | | | |
| P13 | 0.95 | | | | |
| P14 | 0.70 | | | | |
| P15 | 0.40 | | | | |
| Average Chain | 3.68 | 2.68 | 2.13 | 2.28 | 2.49 |
| Length | | | | | |
| % ≧P4 | 33.97 | 22.4 | 7.6 | 9.2 | 12.5 |

These representative examples demonstrate the ability to form solid potassium polyphosphate compositions, such as those having no more than about 30% of the P₂O₅ content as orthophosphate and no more than about 20% of the P₂O₅ content as tetrapoly- and higher polyphosphates. The composition has a K₂O/P₂O₅ mole ratio near 1 (e.g., between about 0.8 and 1.2), and is essentially free of water-insoluble material.

### C. Wet Mix

Table 7 shows representative examples in which 15.83 g polyphosphoric acid at 80.4 wt % P₂O₅ and 50.0 g 60 wt % tetrapotassium pyrophosphate were combined on an equal P₂O₅ basis (overall 1.00 K/P mole ratio) along with water. The resulting mixture having 20.0 g “added water” (30.38 wt %) was split and vacuum dried at two different temperatures. The resulting phosphate species distributions are contrasted against that brought to the reaction by the raw materials (“combined composition”).

TABLE 7
Acid at 80.4 wt % P₂O₅, Vacuum Drying (Series 027)

| | | |
|---|---|---|
| | | Analyzed |
| | | Compositions |
| Phosphate Species, | Combined | Vacuum Drying Temp. |

| % of Total P₂O₅ | Composition | 50° C. | 80° C. |
|---|---|---|---|
| P1 (ortho) | 6.62 | 13.3 | 16.7 |
| P2 (pyro) | 66.59 | 82.3 | 78.4 |
| P3 (tripoly) | 11.83 | 4.0 | 4.3 |
| P4 (tetrapoly) | 7.26 | 0.5 | 0.6 |
| P5 | 4.03 | | |
| P6 | 1.94 | | |
| P7 | 0.85 | | |
| P8 | 0.40 | | |
| P9 | 0.25 | | |
| P10 | 0.25 | | |
| Average Chain | 2.50 | 1.92 | 1.89 |
| Length | | | |
| % ≧P4 | 14.98 | 0.5 | 0.6 |

Preparations identical to those in Table 7 were dried at 160° C. under ambient pressure (i.e., without vacuum) for 6 hours and for 22 hours. The analyzed composition for both preparations resulted in 100.0% of the P₂O₅ content as orthophosphate as compared to Table 7 as a comparative example.

Certain embodiments in Table 8 show representative examples in which polyphosphoric acid at 80.4 wt % P₂O₅ and tetrapotassium pyrophosphate were combined on an equal P₂O₅ basis (overall 1.00 K/P mole ratio) along with water. The resulting mixture was cooled to minus 4° C., which crystallized solids from the solution. The solids were separated by filtration and air dried at ambient temperature. The resulting phosphate species distributions are contrasted against that brought to the reaction by the raw materials (“combined composition”). The example shown in Table 8 demonstrates an embodiment whereby product is separated from the reaction mixture. The example shown in Table 8 also demonstrates the ability to produce a product having a high-assay of pyrophosphate (>90%).

TABLE 8
Acid at 80.4 wt % P₂O₅, Vacuum Filtration and Air Drying of Solids
(Series 067)

| | | |
|---|---|---|
| Phosphate Species, | Combined | Analyzed Composition |

| % of Total P₂O₅ | Composition | Solids | Liquid |
|---|---|---|---|
| P1 (ortho) | 6.54 | 3.0 | 15.6 |
| P2 (pyro) | 67.04 | 94.2 | 57.5 |
| P3 (tripoly) | 11.67 | 2.9 | 13.4 |
| P4 (tetrapoly) | 7.16 | | 7.2 |
| P5 | 3.97 | | 3.5 |
| P6 | 1.91 | | 1.7 |
| P7 | 0.83 | | 0.8 |
| P8 | 0.39 | | 0.4 |
| P9 | 0.25 | | |
| P10 | 0.25 | | |
| Average Chain | 2.49 | 2.00 | 2.36 |
| Length | | | |
| % ≧P4 | 14.75 | — | 13.6 |

The examples shown in Tables 7 and 8 demonstrate the ability to form solid acidic potassium polyphosphate compositions having no more than about 30% of the P₂O₅ content as orthophosphate and no more than about 20% of the P₂O₅ content as tetrapoly- and higher polyphosphates. The compositions have a K₂O/P₂O₅ mole ratio near 1 (e.g., between about 0.8 and about 1.2), and are essentially free of water-insoluble material.

### D. Thermal Route—Comparative Example

A series of experiments were conducted to examine the maximum yield of KAPP achievable by thermal dehydration of MKP and the associated water-insoluble materials level. A source of MKP was used with lower tendency to form water-insoluble materials compared with other sources.

Weight loss was compared with the theoretical weight loss for complete conversion of MKP to KAPP (6.62%), providing the % Conversion of MKP to KAPP (in the absence of large quantities of insoluble material). The % of total P₂O₅ as orthophosphate=(100%−% Conversion). With more than a few percent water-insoluble materials, however, the conversion calculation underestimates the orthophosphate content.

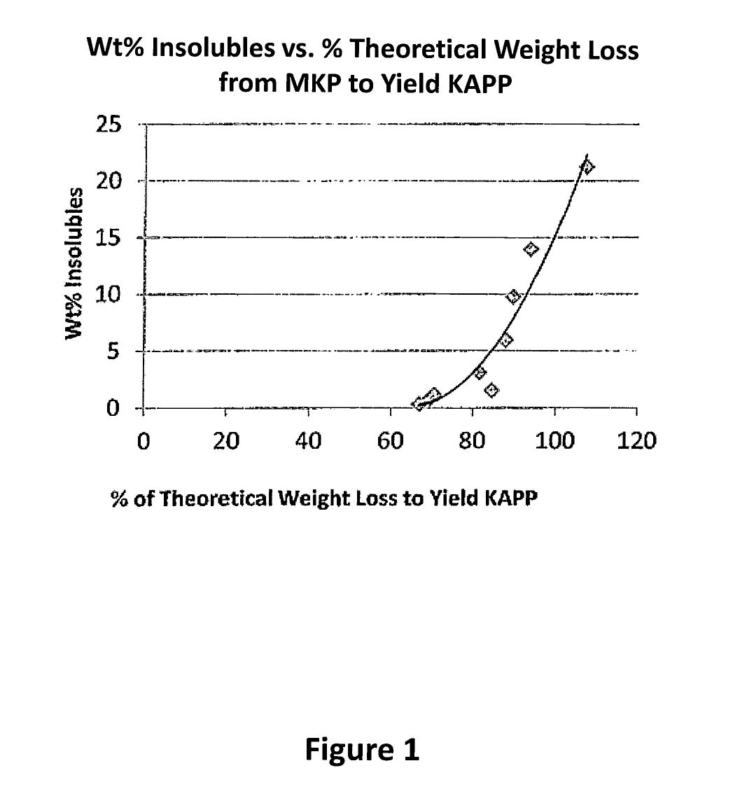

Heating MKP at temperatures≦270° C. resulted in less than about 1% water-insoluble material, however, the % Conversion remained less than about 70%. MKP was heated for various times in the 280° C. to 300° C. temperature range (data provided in Table 9 and FIG. 1). At 280° C., the water-insoluble material level remained near or below 1.0%, even after heating for 5 hours. By increasing the temperature by only 10° C. to 20° C., however, a steep increase in the % water-insoluble material on heating time was observed. Much higher levels of water-insoluble materials form in shorter amounts of time in comparison with heating at or below 280° C. Increasing the heating times in order to achieve incremental conversion above about 70% serves primarily to increase the water-insoluble material content instead.

The % Conversion also increases as a function of temperature for a given heating time. At 280° C., a % Conversion of only about 70% is achievable (i.e., orthophosphate is ≧30% of the total P₂O₅ content). Increasing temperature increases the % weight loss (potential % Conversion), but at the expense of generating water-insoluble materials in excess of 1%.

TABLE 9
Thermal Preparation of KAPP from MKP

| | | | | % Conversion; | |
|---|---|---|---|---|---|
| | | | | % of the | |
| | | | | Theoretical | Water-insoluble |
| | | | MKP Wt. Loss | Weight Loss to | Materials |
| | Furnace Set | Dwell Time at | Upon Heating | Yield KAPP* | in Heated |
| Sample No. | Temp. (° C.) | Set Temp. (hr) | (wt %) | (%) | Sample (wt %) |
| 0454-010-5 | 280 | 2.00 | 4.44 | 67 | 0.31 |
| 0454-014-X | 280 | 3.00 | 4.61 | 70 | 0.82 |
| 0454-014-Y | 280 | 5.00 | 4.68 | 71 | 1.15 |
| 0454-018-290-2 | 290 | 2.00 | 5.41 | 82 | 3.13 |
| 0454-018-290-3 | 290 | 3.00 | 5.94 | 90 | 9.76 |
| 0454-018-290-5 | 290 | 5.00 | 6.23 | 94 | 13.97 |
| 0454-018-300-1 | 300 | 1.00 | 5.61 | 85 | 1.52 |
| 0454-018-300-1.67 | 300 | 1.67 | 5.83 | 88 | 5.95 |
| 0454-014-300 | 300 | 2.00 | 7.10 | 107 | 21.24 |

*Theo. Wt. Loss = 26.62%

In thermal preparations, the formation of water-insoluble materials is strongly linked to the % Conversion. Attempts to push the conversion of MKP to KAPP yield beyond about 70% by employing either higher temperatures and/or longer heating times causes excessive formation of water-insoluble materials.

Sources examined other than shown in Table 9 were observed to form as much as about 8% to 9% water-insoluble materials in 2 hours, or about 20% water-insoluble materials in 3.5 to 5 hours, at 240° C. at about 4.3% to 4.6% weight loss (65-70% of the theoretical weight loss); i.e., well in excess of about 30% of the total P₂O₅ content as orthophosphate.

The breadth and scope of the present disclosure should not be limited by any of the above-described exemplary embodiments, but should be defined only in accordance with the following claims and their equivalents.

1. A potassium polyphosphate composition comprising a mixture of acidic potassium polyphosphates, wherein said mixture of acidic potassium polyphosphates comprises from about 1% to about 30% of its P₂O₅ content as orthophosphate and from about 1% to about 20% of its P₂O₅ content as tetrapoly- and higher polyphosphates, and wherein said composition is a solid composition of matter.

2. The potassium polyphosphate composition of claim 1, wherein the mixture of acidic potassium polyphosphates comprises from about 50% to about 95% of its P₂O₅ content as pyrophosphate.

3. (canceled)

4. The potassium polyphosphate composition of claim 1, wherein the mixture of acidic potassium polyphosphates comprises from about 2% to about 15% of its P₂O₅ content as tripolyphosphate.

5. The potassium polyphosphate composition of claim 1, wherein the combined amount of pyrophosphate and tripolyphosphate in the mixture of acidic potassium polyphosphates is from about 52% to about 97% of the P₂O₅ content of the mixture.

6. The potassium polyphosphate composition of claim 1, wherein the composition has a water-insoluble material content not exceeding about 0.1%, about 0.2%, about 0.3%, about 0.5%, or about 1.0%.

7. (canceled)

8. The potassium polyphosphate composition of claim 1, wherein the composition comprises a K₂O/P₂O₅ molar ratio of from about 0.8 to about 1.2.

9-11. (canceled)

12. The potassium polyphosphate composition of claim 1, wherein the mixture of acidic potassium polyphosphates has an average chain length (n) of between about 1.7 to about 3.3.

13. The potassium polyphosphate composition of claim 1, wherein a 1% by weight solution of said composition in water has a pH of between about pH 3 to about pH 7.

14. A method of preparing a potassium polyphosphate composition, the method comprising reacting a polyphosphoric acid in the range of from about 75 wt % to about 86 wt % P₂O₅ and a potassium polyphosphate salt to form a reaction mixture.

15. The method of preparing a potassium polyphosphate composition of claim 14, wherein the reaction mixture once reacted is the final potassium polyphosphate composition.

16. The method of preparing a potassium polyphosphate composition of claim 14, further comprising drying the reaction mixture to form a solid composition of matter.

17. The method of preparing a potassium polyphosphate composition of claim 16, comprising a step of adding water prior to drying the reaction mixture.

18. The method of preparing a potassium polyphosphate composition of claim 16, wherein the step of drying the reaction mixture is performed under one or more conditions selected from the group consisting of a temperature in a range from about 0° C. to about 100° C., a pressure in a range from about 1 kPa to about 101 kPa, and a period of time in a range from about 1 second to about 24 hours.

19. The method of preparing a potassium polyphosphate composition of claim 16, wherein the step of drying the reaction mixture is performed under a pressure ranging from a complete vacuum to about ambient atmospheric pressure.

20. The method of preparing a potassium polyphosphate composition of claim 18, wherein the temperature is from about 60° C. to about 100° C.

21. The method of preparing a potassium polyphosphate composition of claim 17 wherein the water is added to the polyphosphoric acid and potassium polyphosphate salt at the same time.

22. The method of preparing a potassium polyphosphate composition of claim 17, wherein the water is added to the polyphosphoric acid before reacting the polyphosphoric acid and the potassium polyphosphate salt.

23. The method of preparing a potassium polyphosphate composition of claim 22, wherein the water is added to the polyphosphoric acid within about 20 minutes before reacting the polyphosphoric acid and the potassium polyphosphate salt.

24. The method of preparing a potassium polyphosphate composition of claim 22, wherein the step of adding the water to the polyphosphoric acid includes cooling to negate the heat of dilution.

25. (canceled)

26. The method of preparing a potassium polyphosphate composition of claim 17, wherein a reaction mixture slurry is formed and wherein the method further comprises the step of at least partially separating solids from the reaction mixture slurry prior to drying.

27. The method of preparing a potassium polyphosphate composition of claim 14, wherein the potassium polyphosphate salt comprises tetrapotassium pyrophosphate (TKPP, K₄P₂O₇), potassium tripolyphosphate (KTPP, K₅P₃O₁₀), or a mixture thereof.

28. The method of preparing a potassium polyphosphate composition of claim 14, wherein the potassium polyphosphate salt comprises tetrapotassium pyrophosphate (TKPP, K₄P₂O₇).

29. (canceled)

30. A potassium polyphosphate composition made by the method of claim 14.
